SEQUENCE LISTING (12 sequences)

SEQ ID NO: 1 - DNA, 25 bp, Artificial Sequence
  OLIGONUCLEOTIDE PRIMER
gagcagcaacgagaaataaattggt

SEQ ID NO: 2 - DNA, 22 bp, Artificial Sequence
  PRIMER
gcagacctgtgtaatgcacatg

SEQ ID NO: 3 - DNA, 38 bp, Artificial Sequence
  PRIMER
tgtaagagtgacctaaaaactatacttattctgttaga

SEQ ID NO: 4 - DNA, 25 bp, Artificial Sequence
  PRIMER
ccactgtcctttctcctaacaaact

SEQ ID NO: 5 - DNA, 27 bp, Artificial Sequence
  PRIMER
catcattatcaggtagaggtgacaagt

SEQ ID NO: 6 - DNA, 27 bp, Artificial Sequence
  PRIMER
ctcattgtgtgtttgttttgtcttcct

SEQ ID NO: 7 - DNA, 18 bp, Artificial Sequence
  PRIMER
ctattgttctcgatttct

SEQ ID NO: 8 - DNA, 16 bp, Artificial Sequence
  PRIMER
attgttctcaatttct

SEQ ID NO: 9 - DNA, 20 bp, Artificial Sequence
  PRIMER
aactactacgacctcaaaaa

SEQ ID NO: 10 - DNA, 20 bp, Artificial Sequence
  PRIMER
aactactacgacctcgaaaa

SEQ ID NO: 11 - DNA, 17 bp, Artificial Sequence
  PRIMER
caagggctacttctaac

SEQ ID NO: 12 - DNA, 16 bp, Artificial Sequence
  PRIMER
aagggctacctctaac

(19) United States
(12) Patent Application Publication
(10) Pub. No.: US 2012/0003642 A1
(43) Pub. Date: Jan. 5, 2012

(54) OLIGONUCLEOTIDES AND METHODS FOR DETERMINING SUSCEPTIBILITY TO SOFT TISSUE INJURIES

(75) Inventors: Malcolm Robert Collins, Kirstenhof (ZA); Martin Peter Schwellnus, Pinelands (ZA); Stuart Martin Raleigh, Knighton Leicestershire (GB); William J. Ribbans, Great Houghton Northamptonshire (GB); Roger K. W. Smith, Northampton Northamptonshire (GB)

(73) Assignees: SOUTH AFRICAN MEDICAL RESEARCH COUNCIL, Cape Town (ZA); THE UNIVERSITY OF NORTHAMPTON, Northamptonshire Northampton (GB); UNIVERSITY OF CAPE TOWN, Cape Town (ZA)

(21) Appl. No.: 13/127,668

(22) PCT Filed: Nov. 4, 2009
(86) PCT No.: PCT/IB09/54892

§ 371 Date: Sep. 19, 2011

(30) Foreign Application Priority Data
Nov. 4, 2008 (ZA) 2008/09435

(51) Int. Cl. C12Q 1/68; C07H 21/04

(52) U.S. Cl. 435/6.11; 536/24.31; 536/24.33

(57) ABSTRACT

A method of determining in a subject a predisposition to, or increased risk for, developing a tendon, ligament, or other soft tissue injury or pathology, the method comprising the step of screening the subject for the presence of at least one poly-morphism in at least one gene family selected from the group consisting of any one or more of: the matrix metallo-protease (MMP) family, the collagen family, including the COL5A1 and COL12A1 genes, the glycoprotein family, including the TNC and COMP genes, and derivatives thereof, which polymorphism is a polymorphism which results in a modified, augmented, or gated interaction with other members of the gene families mentioned herein, when compared to a wild-type interaction.

### FIELD OF THE INVENTION

THIS INVENTION relates to risk factors associated with tendon and ligament injuries. More particularly, this invention relates to molecular markers useful in determining increased susceptibility or risk of a subject in developing a tendon, ligament, and/or soft tissue pathology; to an assay for use in determining an increased susceptibility or risk in a subject in developing a tendon, ligament and/or soft tissue pathology; to a method for diagnosing or determining an increased risk or susceptibility in a subject in developing a tendon, ligament and/or soft tissue pathology; and to a kit for use in determining an increased risk or susceptibility of a subject in developing tendon and/or ligament pathologies.

### BACKGROUND OF THE INVENTION

There is a spectrum of pathologies that can affect soft tissues, tendons and ligaments, as well as their surrounding structures'. As an example, Achilles Tendinopathy (AT) is one of these pathologies which is a painful and degenerative condition that affects subjects who participate in a range of sporting pursuits, as well as occurring in the less physically active^{2,3}. Acute spontaneous rupture is another common pathology that can affect the Achilles tendon, particularly in the middle-aged, male athlete. Injury to the anterior cruciate ligament (ACL) is an example of a common ligament injury. It has been described as one of the most severe injuries sustained in a sporting population. Participants of sports which involve a sudden deceleration or change in direction are particularly at risk of rupturing their ACL. A number of intrinsic and extrinsic factors have been implicated in raising the risk of both tendon and ligament pathologies⁵.

In recent years evidence has emerged that Achilles tendon injuries, ACL injuries, as well as rotator cuff injuries and shoulder dislocations have a genetic component. As such, it has been shown that a GT repeat variant within the gene that encodes the tenascin C protein (TNC), a key constituent of tendon which is regulated by mechanical loading^{6,7}, is associated with Achilles tendinopathy and rupture⁸. In addition to the TNC gene, polymorphisms within the 3′-untranslated region of the COL5A1 gene have also been associated with Achilles tendinopathy in both South African⁹ and, as more recently shown, Australian populations¹⁰. This polymorphism has also recently been shown to be associated with ACL injuries in female athletes. Interestingly, variations within the Sp1 binding site of another collagen gene, viz. COL1A1, does not appear to independently associate with Achilles tendon pathology¹¹, nor do polymorphisms within the related COL14A1 and COL12A1 genes¹².

In addition to the TNC and COL5A1 genes, it may well be that predisposition to tendon, ligament, and other soft tissue injuries may be associated with other genes. Such genes may encode proteins with regulatory roles in maintaining extracellular matrix (ECM) homeostasis. It is an object of the present invention to locate other genes or polymorphs which may be involved in tendon, ligament and/or other soft tissue pathologies. It is a further object of the invention to locate possible areas within so-called MMP genes which may be associated with tendon and ligament pathologies. A further object of the invention is to determine whether MMP3 may be involved in tendon and ligament pathologies by interacting with other genes or gene products, and to provide diagnostic assays to identify subjects or individuals at risk of developing tendon, ligament, and other soft tissue injuries and pathologies, including, but not limited to, Achilles tendon pathologies and ACL ruptures. A further object of the invention is to determine whether MMP genes interact with the specific markers in genes that encode other protein components, (such as collagens, proteoglycans, and glyoproteins, of connective tissue) in modulating the risk of soft tissue injuries.

### SUMMARY OF THE INVENTION

Broadly, according to one aspect of the invention, there is provided a method of determining in a subject a predisposition to, or increased risk for, developing a tendon, ligament, or other soft tissue injury or pathology, the method comprising the step of screening the subject for the presence of at least one polymorphism in at least one gene family selected from the group consisting of any one or more of:
the matrix metallo-protease (MMP) family;
the collagen family, including the COL5A1 and COL12A1 genes;
the glycoprotein family, including the TNC and COMP genes; and
derivatives thereof,
   which polymorphism is a polymorphism which results in a modified, augmented, or mitigated interaction with other members of the gene families mentioned herein, when compared to a wild-type interaction.

The collagen gene family may include the COL5A1 and COL12A1 genes. The glycoprotein gene family may include the TNC and COMP genes.

More specifically, the method may include the step of detecting or screening for the presence of a polymorphism in the matrix metallo-protease 3 (MMP3) gene which has modified, augmented, or mitigated interaction with a COL5A1 polymorphism product, when compared to a wild-type interaction. More particularly, the MMP3 polymorphism may be a polymorphism which has a modified, augmented, or mitigated interaction with the rs12733 COL5A1 polymorphism, and/or any other linked polymorphism, and the product encoded thereby.

According to another aspect of the invention, there is provided a molecular marker for use in diagnosing a predisposition to, or increased risk for, developing tendon, ligament, or other soft tissue pathology or injury in a subject, the molecular marker comprising at least one isolated nucleic acid fragment derived from an MMP3 gene, flanking sequences thereof, cis-regions associated therewith, 5′UTR regions, 3′UTR regions thereof, sequences complementary thereto, sequences which can hybridize under strict hybridization conditions thereto, and functional discriminatory truncations thereof. The molecular marker may be DNA-based, RNA-based, or other combinations of nucleic acids or modified bases.

The molecular marker may be a part of, or a fragment derived from, an MMP3 gene, the fragment being between 10 and 40, preferably between 15 and 35, more preferably between 20 and 30 nucleic acids in length, and which hybridizes under stringent hybridization conditions to at least a portion of the MMP3 gene. This may include sequences complementary to the marker, and sequences having substitutions, deletions or insertions, sequences which can hybridize under strict hybridization conditions thereto, and functional discriminatory truncations thereof.

In one embodiment, the molecular marker is a polymorphic marker, preferably an SNP. The SNP may be any one or more SNPs selected from the group consisting of rs591058, rs679620, and rs650108, together with any other SNP closely linked (i.e. which is in high linkage disequilibrium) with any of the three specific SNPs listed above.

More particularly, the SNPs may be selected from the group consisting of:
(i) rs679620, an A/G transition at nucleotide position 28 within exon 2, E45K;
(ii) rs591058, a T/C transition at nucleotide position 1547 within intron 4; and
(iii) rs650108, a G/A transition at position 495 within intron 8.

More particularly, the molecular marker may be, or may be detectable using, any one or more isolated oligonucleotides selected from the group comprising:

SEQ. ID. NO. 1:
GAGCAGCAACGAGAAATAAATTGGT;
SEQ. ID. NO. 2:
GCAGACCTGTGTAATGCACATG;
SEQ. ID. NO. 3:
TGTAAGAGTGACCTAAAAACTATACTTATTCTGTTAGA;
SEQ. ID. NO. 4:
CCACTGTCCTTTCTCCTAACAAACT;
SEQ. ID. NO. 5:
CATCATTATCAGGTAGAGGTGACAAGT;
SEQ. ID. NO. 6:
CTCATTGTGTGTTTGTTTTGTCTTCCT;

sequences complementary thereto, sequences which can hybridize under stringent hybridization conditions thereto, and functional discriminatory truncations thereof.

Accordingly, the invention extends to a primer or oligonucleotide sets for use in detecting or diagnosing a predisposition to, or increased risk for, developing tendon, ligament, or other soft tissue pathologies or injuries in a subject, the primer or oligonucleotide sets comprising isolated nucleic acid sequences selected from the group consisting of:

Set 1:
SEQ. ID. NO. 1:
GAGCAGCAACGAGAAATAAATTGGT;
SEQ. ID. NO. 2:
GCAGACCTGTGTAATGCACATG;
Set 2
SEQ. ID. NO. 3:
TGTAAGAGTGACCTAAAAACTATACTTATTCTGTTAGA;
SEQ. ID. NO. 4:
CCACTGTCCTTTCTCCTAACAAACT;
Set 3
SEQ. ID. NO. 5:
CATCATTATCAGGTAGAGGTGACAAGT;
SEQ. ID. NO. 6:
CTCATTGTGTGTTTGTTTTGTCTTCCT;
and

sequences complementary thereto, sequences which can hybridize under stringent hybridization conditions thereto, and functional discriminatory truncations thereof.

In addition, the molecular marker may comprise any one or more isolated nucleic acid sequences selected from the group consisting of:

SEQ. ID. NO. 7:
CTATTGTTCTCGATTTCT;
SEQ. ID. NO. 8:
ATTGTTCTCAATTTCT;
SEQ. ID. NO. 9:
AACTACTACGACCTCAAAAA;
SEQ. ID. NO. 10:
AACTACTACGACCTCGAAAA;
SEQ. ID. NO. 11:
CAAGGGCTACTTCTAAC;
SEQ. ID. NO. 12:
AAGGGCTACCTCTAAC;
and

fragments thereof, sequences complementary thereto, sequences which can hybridize under stringent hybridization conditions thereto, and functional discriminatory truncations thereof.

According to a still further aspect of the invention, there is provided an isolated nucleic acid molecule for detecting at least one SNP provided hereinbefore, wherein the nucleic acid molecule comprises less than 40, less than 30, less than 20, or even preferably less than 10 contiguous nucleotides selected from the group consisting of SEQ ID NOS 1 to 12 and fragments, complementary sequences, sequences which can hybridize under stringent hybridization conditions thereto, and functional discriminatory truncations thereof.

The invention extends also to a detection reagent capable of detecting one or more single nucleic acid polymorphisms selected from the group consisting of the SNPs listed hereinbefore, fragments thereof, sequences complementary thereto, sequences which can hybridize under stringent hybridization conditions thereto, and functional discriminatory truncations thereof.

The invention extends to the use of the sequences and/or markers of the invention in other assays, such as RFLPs and AFLPs.

According to another aspect of the invention, there is provided a diagnostic assay comprising any one or more of the markers described hereinbefore, fragments thereof, sequences complementary thereto, sequences which can hybridize under stringent hybridization conditions thereto, and functional discriminatory truncations thereof.

According to yet another aspect of the invention, there is provided a method of determining a predisposition for, or increased risk of, developing a tendon, ligament and/or soft tissue pathology or injury in a subject, the method comprising the steps of screening a subject for a polymorphism in an MMP3 gene.

The polymorphism may be any one of more of the polymorphisms listed hereinbefore, polymorphisms in high linkage disequilibrium with the listed polymorphisms, or a polymorphism detectable using any one or more of the sequences listed hereinbefore, fragments thereof, sequences complementary thereto, sequences which can hybridize under stringent hybridization conditions thereto, and functional discriminatory truncations thereof.

The method may include the additional steps of:
providing a tissue sample from a subject;
extracting nucleic acid from the sample;
amplifying selected regions of the nucleic acid using any one or more of the molecular markers selected from the group consisting of: SEQ. ID. NOs 1 to 2, thereby to obtain amplified nucleic acid fragments; and
screening the amplified nucleic acid fragments for the presence of the polymorphisms listed hereinbefore.

According to another aspect of the invention, there is provided use of a molecular marker of the invention in diagnosing a predisposition to a soft tissue pathology in a subject.

According to a still further aspect of the invention, there is provided a kit for use in diagnosing a predisposition to a soft tissue pathology in a subject, the kit comprising:
any one or more of the molecular markers selected from the group consisting of:
SEQ. ID. Nos 1 to 12; and
suitable reaction media.

The kit may further include any one or more of reagents, such as buffers, DNases, RNAses, polymerases, instructions, and the like.

The molecular markers may be any one or more markers selected from the markers listed hereinbefore.

The soft tissue may be a connective tissue injury, and may include tendon and/or ligament injuries such as, for example, Achilles tendon, rotator cuff tendons, patellar tendon, shoulder ligament, knee ligament and ankle ligament pathologies. The sample may comprise an animal tissue or blood sample, such as a human tissue or blood sample.

Further features of the invention will now be described with reference to the following non-limiting examples and figures.

### DRAWINGS

In the drawings:

FIG. 1 shows: A schematic representation of the exon (rectangles) and intron (horizontal lines) boundaries of the MMP3 (matrix metallopeptidase 3) gene, which is located in the negative orientation on chromosome 11. The translated regions of the exons are shown in solid colour, while the untranslated regions (UTRs) are clear. Exon numbers are as indicated. The chromosomal location and the size of the gene are given in brackets. Single nucleotide polymorphisms (SNPs) with high heterozygous frequencies (>20%) identified from databases hosted by the international HapMap project are annotated (clear boxes). In addition, three synonomous, one non-synonomous, and a promoter (rs3025058, −/T) SNP identified from databases hosted by the National Centre for Biotechnology Information (NCBI) are also annotated (grey boxes). The three SNPs of the present invention are annotated below the gene as clear boxes. Accession numbers and base changes are indicated for all the SNPs. The minor alleles and, unless otherwise indicated, their frequencies from the HapMap CEU population, are also indicated. Where applicable the amino acid change and number of the exonic SNPs, as well as the nucleotide position of the two SNPs within the promoter region, are indicated. ¹The complementary nucleotides are given in the databases and used in the present document to avoid confusion. ²Since frequency data was not available for the HapMap CEU population, minor allele frequency data from other available European population data within the NCBI databases are indicated. ³Also referred to as the −1171 5A/6A polymorphism.

FIG. 2 shows: The relative genotype (A to C) and allele (D to F) frequencies of the MMP3 single nucleotide polymorphisms (SNPs) rs679630 (A and D), rs591058 (B and E) and rs650108 (C and F) for an asymptomatic control (CON, clear bars); chronic Achilles tendinopathy (TEN, solid bars); and Achilles rupture (RUP, hatched bars) groups. (A) rs679620: TEN vs CON, P=0.031; RUP vs CON P=0.666; (B) rs591058: TEN vs CON, P=0.065; RUP vs CON P=0.734. (C) rs650108: TEN vs CON, P=0.093; RUP vs CON P=0.627. (D) rs679620: TEN vs CON, P=0.037; RUP vs CON P=0.500. (E) rs591058: TEN vs CON, P=0.051; RUP vs CON P=0.592. (F) rs650108: TEN vs CON, P=0.368; RUP vs CON P=0.754. The asterisks and solid lines represent specific genotype (A) GG, P=0.010; (B) CC, P=0.023; (C) AA, P=0.043; or allele (D) G, P=0.0.037; significant differences between the TEN and CON groups. The number (N) of subjects (A to C) or alleles (D to F) are indicated in parenthesis.

FIG. 3 shows: (A) Linkage disequilibrium (LD) structure of the three single nucleotide polymorphisms (SNPs) rs679620, rs591058, and rs650108 within the MMP3 gene. The LD map was constructed using the combined genotype data from the two Achilles tendon pathology and control groups. As indicated in the grey scale key, the strength of LD between any two SNPs is indicated by the colour of the cells. The D′ values, sample sizes (N) and the coefficient of correlations (r) between pairs of markers are given within each cell. A positive correlation (r) represents major alleles being associated with each other, while a negative correlation (r) means the major allele of the one SNP is associated with the minor allelle of the other and vice versa. (B) Inferred haplotype frequency distributions from MMP3 rs679620, rs591058, and rs650108 in the control group (CON, clear bars) and Achilles tendinopathy (TEN, solid bars) group. Global P=0.144. The asterisk and solid line marks the significant difference (P=0.038) between the ATG haplotype pair. The number (N) of subjects in each group is in parentheses.

FIG. 4 shows: The relative distribution of the allele combinations of MMP3 SNP rs679620 (A/G) and COL5A1 SNP rs12733 (C/T) for the control (CON, clear bars) and Achilles tendinopathy (TEN, solid bars) groups. Global stat=10.4, df=3 and P=0.016. The asterisk and solid line marks the significant difference between the allele combination pairs. AC, P=0.002 and GT, P=0.006. The number (N) of subjects in each group is in parenthesis.

### DETAILED DESCRIPTION OF AN EMBODIMENT OF THE INVENTION

For the purposes of this specification, a “polymorphism” may include a change or difference between two related nucleic acids. A “nucleotide polymorphism” refers to a nucleotide which is different in one sequence when compared to a related sequence when the two nucleic acids are aligned for maximal correspondence. A “probe” or “molecular marker” is an RNA sequence(s) or DNA sequence(s) or analogues, modified versions, or the complement of the sequences shown. This may include a “genetic marker”, which is a region on a genomic nucleic acid mapped by a molecular marker or probe. A “probe” is a composition labelled with a detectable label. A “probe” is typically used herein to identify a marker nucleic acid. A polynucleotide probe is usually a single-stranded nucleic acid sequence that can be used to identify complementary nucleic acid sequences, or may be a double- or higher order-stranded nucleic acid sequence which can be used to bind to, or associate with, a target sequence or area, generally following denaturing. The sequence of the polynucleotide probe may or may not be known. An RNA probe may hybridize with its corresponding DNA gene, or to a complementary RNA, or to other type of nucleic acid molecules. As used herein the term “functional discriminatory truncations” mean nucleic acid sequences, modified nucleic acid sequences, or other nucleic acid variants which, although they are truncated forms of sequences presented herein or variants thereof, can still bind in a discriminatory manner to target gene or nucleic acid sequences described herein and forming part of the present invention. The terms “isolated” or “biologically pure” refer to material which is substantially or essentially free from components which normally accompany it as found in its native state. An “amplified mixture” of nucleic acids includes multiple copies of more than one (and generally several) nucleic acids. “Stringent hybridization conditions” in the context of nucleic acid hybridization are sequence dependent and are different under different environmental parameters. Generally, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Highly stringent conditions are selected to be equal to the Tₘ point for a particular probe. An example of stringent wash conditions for, say, a Southern blot of such nucleic acids is a 0.2×SSC wash at 65° C. for 15 minutes. Such a high stringency wash may be preceded by a low stringency wash to remove background probe signal. An example of a low stringency wash is 2×SSC at 40° C. for 15 minutes. In general, a signal to noise ratio of 2× (or higher) than that observed for an unrelated probe in the particular hybridization assay indicates detection of a specific hybridization event. For highly specific hybridization strategies such as allele-specific hybridization, an allele-specific probe is usually hybridized to a marker nucleic acid (e.g., a genomic nucleic acid, an amplicon, or the like) comprising a polymorphic nucleotide under highly stringent conditions.

### Materials and Methods

### Subjects

One hundred and fourteen Caucasian subjects diagnosed with Achilles tendon injuries, including 75 with chronic Achilles tendinopathy (TEN) using clinical criteria and 39 with partial (N=3) or complete ruptures of the Achilles tendon (RUP), were recruited for this study from the medical practice at the Sports Science Institute of South Africa and other clinical practices within the greater Cape Town area of South Africa. Rupture of the Achilles tendon was confirmed during surgery or by imaging. Ten of the subjects in the RUP group had a history of tendinopathy. An additional, 98 apparently healthy, unrelated, Caucasian subjects without any history of symptomatic Achilles tendon injuries were recruited as controls (CON). The inclusion and exclusion criteria of the participants have been described previously^{8,9}.

Prior to participation in this study, subjects gave informed written consent and completed medical history questionnaire forms. This study was approved by the Research Ethics Committees of the Faculty of Health Sciences within the University of Cape Town, South Africa and the University of Northampton, England.

### DNA Extraction and Single Nucleotide Polymorphism (SNP) Selection

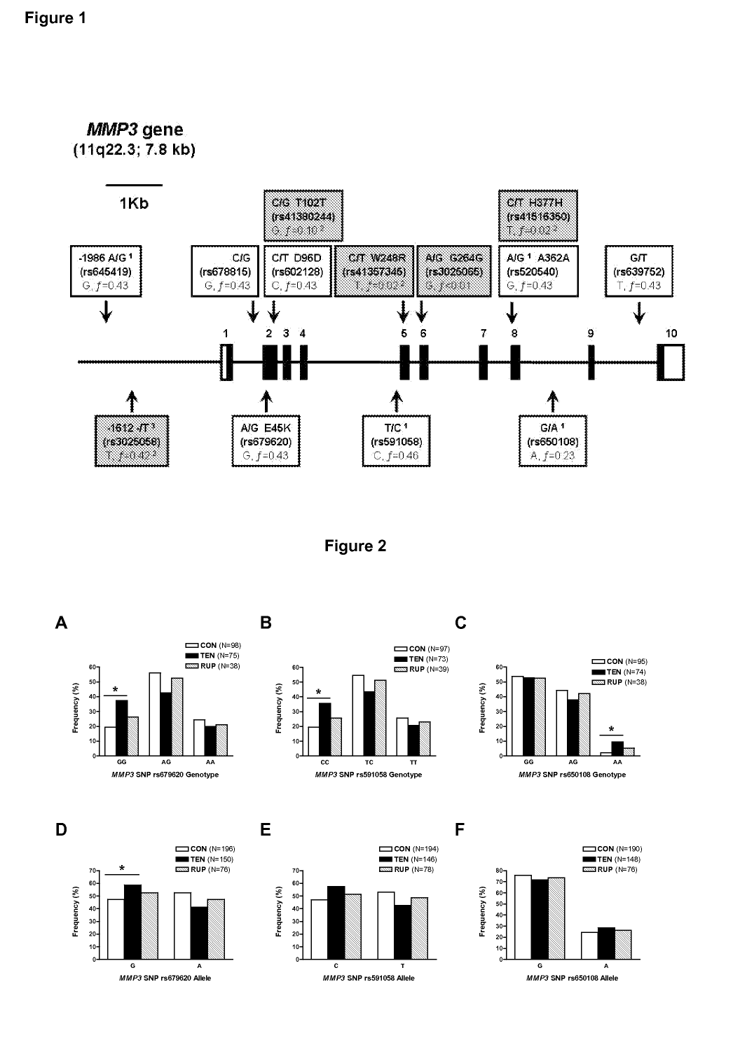

DNA was extracted using the procedure described by Lahiri and Nurnberg²⁰ and modified by Mokone et al.⁸ Single nucleotide polymorphisms (SNPs) within the MMP3 gene and its 5′-flanking sequence were identified from databases hosted by the National Centre for Biotechnology Information (NCBI) (www.ncbi.nlm.nih.gov) and the International HapMap project (www.hapmap.org). The MMP3 gene is transcribed in the negative orientation resulting in the mRNA sequence corresponding to the bottom DNA strand. As indicated in FIG. 1, the complementary nucleotides are given in the databases for some of the SNPs. These complementary nucleotides are used in this manuscript to avoid confusion. Seven exonic SNPs were identified and annotated onto a schematic diagram (FIG. 1). Two of these SNPs were non-synonomous (i.e. SNPs which change the amino acid sequence in the gene product), of which only one, rs679620 (E45K) within exon 2, has a high heterozygous frequency and could therefore be potentially informative in genetic case-control association studies. Two of the five synonomous SNPs, rs602128 (D96D) within exon 2 and rs520540 (A362A) within exon 8, also have a high heterozygous frequency and were therefore considered potentially informative. From the databases hosted by the International HapMap project it was found that these three exonic SNPs formed a single haplotype block together with four informative (high heterozygosity) intronic SNPs that spanned the entire gene and a single informative SNP (rs645419) at −2 kb within the promoter region (FIG. 1). The functional 5A/6A (rs3025058, −/T) polymorphism, which is often used in association studies involving the MMP3 gene ^{16,17}, is located downstream of SNP rs645419. Four major haplotypess which contained these eight potentially informative SNPs were identified using haploView version 4.1 ²² and the HapMap CEU data (release 22). The same four haplotypes with similar frequencies were also identified when only three of the eights SNPs were analysed. In this study, the MMP3 gene was therefore genotyped for the following SNPs: (1) rs679620, an A/G transition at nucleotide position 28 within exon 2, E45K; (2) rs591058, a T/C transition at nucleotide position 1547 within intron 4; and (3) rs650108, a G/A transition at position 495 within intron 8.

### MMP3 SNP Genotyping

DNA samples were genotyped for all three MMP3 variants using fluorescence-based Taqman® technology (Applied Biosystems, Foster City, Calif., USA). Allele specific probes and flanking primer or oligonucleotide sets (Table 1) were used along with a pre-made PCR mastermix containing ampliTaq DNA polymerase Gold (Applied Biosystems, Foster City, Calif., USA) in a total reaction volume of 25 μl. PCR consisted of a 10 minute heat activation step (95° C.) followed by 40 cycles of 15 s at 92° C. and 1 minute at 60° C. PCR was performed on an MJ Miniopticon thermocycler (BioRad, UK) and genotypes were determined by endpoint fluorescence using MJ Monitor analysis software (version 3.1).

### Statistical Analyses

Assuming an allele frequency of 0.43 (observed for MMP3) in the CON group, group sizes of 97 in each group would be adequate to detect an allelic odds ratio of at least 2.25 at a power of 80% and significance level of 5%. Linear and logistic regression models were used to assess differences between the characteristics of the TEN, RUP and CON groups for quantitative and categorical data, respectively. Logistic regression was used to compare the combined, as well as the separate (TEN and RUP) Achilles tendon cases to the control groups, with respect to genotype, allele and haplotype frequencies. Two different methods were used to assess gene-gene interaction between MMP3 and COL5A1 (SNP rs12733). Allele combinations consisting of the markers on the two different genes were constructed and their association with case-control status was tested. The OR MDR (odds ratio based multifactor dimensionality reduction) method²³ was also used to select from the pair of SNPs (from all SNPs) with the strongest association with tendinopathy. The quantitative measure of disease risk being analysed is commonly referred to as an OR, and it represents the estimated relative risk of disease with a specific combination of genotypes. Data was analysed using the freely available programming language R (www.r-project.org), specifically packages DGC-genetics (LD, Hardy-Weinberg, genotype and allelic association), haplo.stats (inferred haplotype association) and ormdr (interaction between loci on case-control status). Pass2008 (www.ncss.com) was used for sample size calculation.

### Results

### Subject Characteristics

The TEN, RUP and CON groups were similarly matched for age, height, gender and country of birth (Table 2). The age of the TEN and RUP groups were the age of initial onset of symptoms of Achilles tendon injury, which were on average 7.8±8.0 and 7.5±8.9 years prior to recruitment in this study respectively. The reported weights were also as at time of recruitment, not at time of onset of injury. The TEN and RUP groups were on average significantly heavier with corresponding higher BMI than the CON group. In addition, the RUP group was also on average significantly heavier, with corresponding higher BMI, than the TEN group. There were no MMP3 single nucleotide polymorphism genotype effects on any of the subject characteristics (results not shown).

Twenty six (38.2%) and 5 (13.5%) of the subjects were diagnosed with bilateral chronic Achilles tendinopathy and Achilles rupture respectively. Multiple Achilles tendon injuries (greater than one) were documented in 16 (22.9%) and 13 (35.1%) of the TEN and RUP subjects respectively. Forty four percent of the TEN and 35% of the RUP subjects reported either a bilateral and/or multiple Achilles tendon injury.

### Genotype and Allele Frequencies

There were no significant differences in the genotype distributions of SNPs rs679620 (P=0.064), rs591058 (P=0.117) and rs650108 (P=0.132) between the Achilles tendon pathology (combined TEN and RUP) and control groups (see FIGS. 2A to 2C). Similarly, there were no significant differences found in the allele distributions of SNPs rs679620 (P=0.057), rs591058 (P=0.083), and rs650108 (P=0.404) between the combined pathology and control groups (FIGS. 2D to 2F). Since differences had been detected in genotype distributions between subjects with chronic Achilles tendinopathy or Achilles tendon ruptures¹², the Achilles tendon pathology group was sub-divided into tendinopathy (TEN) and rupture (RUP) sub-groups. Surprisingly, there were significant differences in the distribution of the genotype (P=0.031) (FIG. 2A) and allele (P=0.037) (FIG. 2D) frequencies of the MMP3 rs679620 SNP between the CON and TEN groups. The GG genotype was significantly over-represented in TEN group (37.3%, N=28) when compared to the CON group (19.4%, N=19) (P=0.010, OR=2.5, 95% CI 1.2-4.9). The differences in the genotype (FIGS. 2B and 2C) or allele (FIGS. 2E and 2F) frequency distributions of the MMP3 rs591058 (genotype P=0.065 and allele P=0.051) and rs650108 (genotype P=0.093 and allele P=0.368) between the CON and TEN groups were not significant. However, the CC genotype of SNP rs591058 was over-represented in the TEN (35.6%, N=26) compared to the CON (19.6%, N=19) (P=0.023, OR=2.3, 95% CI 1.1-4.5), while the AA genotype of SNP rs650108 was over-represented in the TEN (9.5%, N=7) compared to the CON (2.1%, N=2) (P=0.043, OR=4.9, 95% CI 1.0-24.1). Similar results were obtained when the 10 RUP subjects with a history of tendinopathy were included in the analysis as part of the TEN group (results not shown).

There were, however, no significant differences in the distribution of the genotype (rs679620, P=0.666; rs591058, P=0.734; and rs650108, P=0.627) (FIGS. 2A, 2B and 2C) and allele (rs679620, P=0.527; rs591058, P=0.604; and rs650108, P=0.840) (FIGS. 2D, 2E, 2F) frequencies of the three MMP3 SNPs between the CON and RUP groups. The three MMP3 SNP genotype distributions within the CON, TEN and RUP groups were in Hardy-Weinberg equilibrium. Although there was a reduction in statistical power, similar genotype distributions were nevertheless observed when only the South African-born subjects were analysed (results not shown).

### Linkage Disequilibrium (LD) and Inferred Haplotype Analysis of MMP3

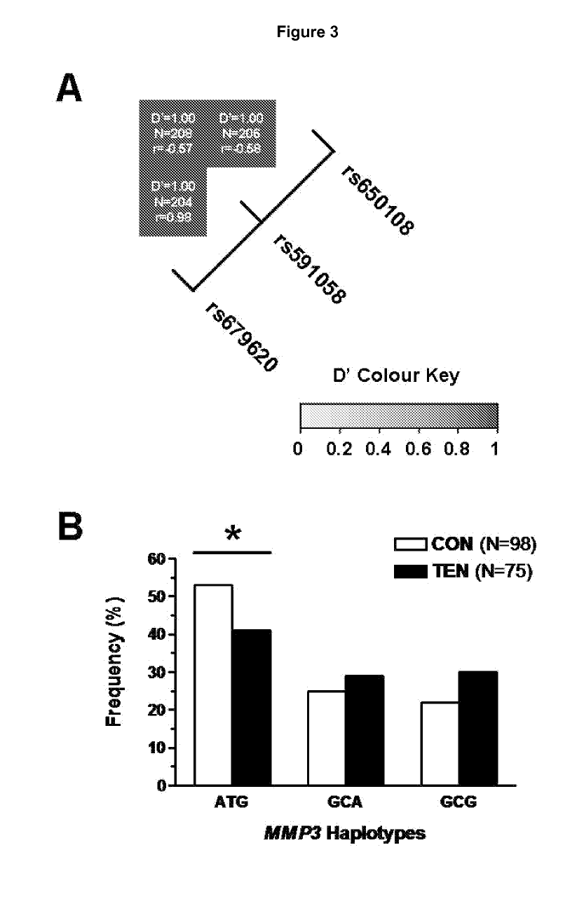

The two SNPs rs679620 (A/G) and rs591058 (T/C) were shown to be in almost perfect LD. Allele G in the former corresponds to allele C in the latter, with only one heterozygous subject/individual for rs679620 being TT for rs591058. The D′ measure is 1 (P<0.001) and the coefficient of correlation, r, is 0.98. Both SNPs are also in high LD with rs650108, D′=1 (P<0.001). The coefficient of correlation of rs650108 is −0.57 and −0.58 with rs679620 and rs591058 respectively (FIG. 3A). Similar values were obtained when the CON, TEN and RUP subjects were analysed separately (results not shown).

Only the AT and GC haplotypes were inferred with a frequency greater than 1% from SNPs rs679620 and rs591058. Since the GG and CC genotypes of these SNPs were over-represented in the Achilles tendinopathy group (FIGS. 2A and 2B), the GC haplotype was over-represented in this group (47% CON vs 59% TEN, P=0.031). As both these MMP3 SNPs were also in high LD with rs650108 (FIG. 3A), only three of the eight possible haplotypes (ATG, GCA and GCG) containing the three SNPs were inferred with a frequency more than 1%. The ATG haplotype (53% CON vs 41% TEN) was significantly under-represented in the TEN group (P=0.038) (FIG. 3B).

### MMP3 and COL5A1 Gene-Gene Interaction

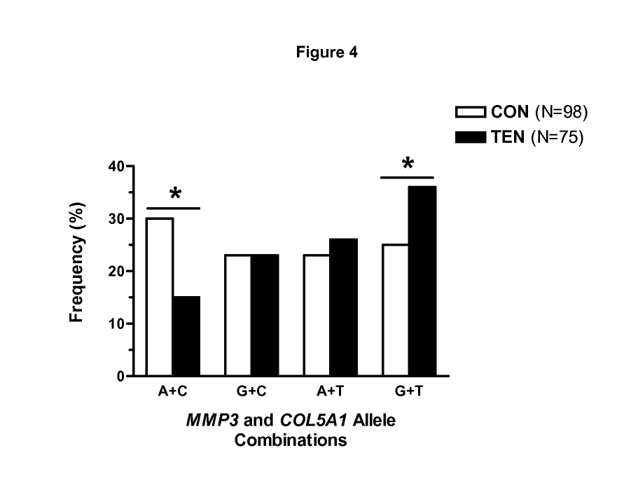

September et al.¹⁰ have previously shown that the CC genotype of the COL5A1 BstUI RFLP (rs12722) was under-represented in patients with chronic Achilles tendinopathy. MMP3 SNP rs679620 together with the COL5A1 SNP formed the best pair of genotypes for estimating risk for Achilles tendinoapthy. The genotype pairs together with their frequencies and estimated risk are summarised in Table 3. The MMP3 rs679620 A allele (AA or AG genotype) combined with the COL5A1 rs12722 CC genotype had the lowest risk for Achilles tendinopathy. All the possible A and C allele combinations were associated with the lowest risk. In support of this, the MMP3 rs679620 G/A and COL5A1 rs12722 C/T allele combinations (pseudo-haplotypes) were significantly associated with TEN and CON status (global-stat=10.4, df=3, P=0.016) (FIG. 4). The A+C allele combination was significantly associated with the controls (30% CON vs 15% TEN, P=0.002), while the G+T allele combination was significantly associated with TEN (25% CON vs 36% TEN, P=0.006). To date, there have been no genetic association studies of which the Applicant is aware about the role of MMPs²⁴ and MMP3²⁵ genes in Achilles tendinopathy, or rupture. Accordingly, the results presented herein show the first evidence that sequence variations within the MMP3 gene are associated with AT. Moreover, these data also demonstrate that a significant interaction exists between the exonic MMP3 SNP (rs679620) and the COL5A1 3′-untranslated region (rs12722) polymorphism and risk of AT.

All three MMP3 polymorphisms (rs679620, rs591058 and rs650108) investigated in this disclosure have been found to be associated with AT. As single loci, the rs679620 polymorphism, GG genotype, and rs591058 polymorphism, CC genotype, co-segregate with Achilles tendinopathy with odds ratios of 2.5 and 2.3 respectively. The rs650108 SNP, AA genotype has a higher odds ratio (4.9), but only 7 cases possessed this genotype due to its low allele frequency in the population. When analysed as an inferred haplotype, the ATG sequence combination of these three SNPs is significantly under-represented in AT cases compared to controls and it may be that this haplotype protects against the development of AT.

Reduction in MMP3 RNA or protein is likely to result in increased proteoglycans²⁷ which may result in degenerative tendons. Allelic association to a trait or disease does not necessarily infer cause. However, the rs679620 variant of MMP3 is a non-synonymous polymorphism. Specifically, a glutamate residue is coded for by inclusion of the G allele (GAA codon) at the rs679620 loci, while a lysine residue is encoded for by the A allele (AAA codon). Although both residues are polar, the glutamate side chain is negatively charged compared to the positive charge on lysine³¹. The residue sits at position 45 from the start of the polypeptide chain³² and the first 82 residues, incorporating the propeptide,³³ are cleaved by a proteinase during the processing of proMMP3 into mature MMP3³⁴. Appropriate removal of the propeptide may have some dependency on the presence of either a Lys or Glu at position 45 and hence may influence downstream function of the mature MMP3 enzyme. This association may be due to genetic linkage between the non-synonymous MMP3 polymorphism (rs679620) and other polymorphisms within the MMP3 gene and flanking sequences. In support of this, the three SNPs investigated in this study, which spanned most of the gene, were in high linkage disequilibrium (D′=1) with each other. Of the 23 MMPs in humans, 9 of their genes form a cluster on the long arm of chromosome 11. The MMP3 gene is part of this cluster and due to the nature of genetic association studies it cannot be excluded with all certainty that one of the other 8 MMP genes are involved in the pathogenesis of AT.

The data presented herein demonstrate that the MMP3 variants investigated in this study interact with the COL5A1 rs12733 polymorphism in modifying the risk of AT. Although AT is likely to be a complex condition involving a number of gene-gene and gene-environment interactions³⁵, there have, to the Applicant's knowledge, been no such reports of a gene-gene interaction that relates to increased risk of AT. As COL5A1 is a substrate for MMP3³⁶, subjects or individuals that carry risk variants within both of these genes may have disrupted interactions between type V collagen and MMP3 during catalysis leading to a heightened risk of AT.

In one embodiment, the molecular marker is a polymorphic marker, preferably an SNP. The SNP is any one or more SNPs selected from the group consisting of rs591058, rs679620, and rs650108, together with any other SNP closely linked (i.e. which is in high linkage disequilibrium) with any of the three specific SNPs listed above.

More particularly, the SNPs is selected from the group consisting of:
(i) rs679620, an A/G transition at nucleotide position 28 within exon 2, E45K;
(ii) rs591058, a T/C transition at nucleotide position 1547 within intron 4; and
(iii) rs650108, a G/A transition at position 495 within intron 8.

More particularly, the molecular marker is, or is detectable using, any one or more isolated oligonucleotides selected from the group comprising:

SEQ. ID. NO. 1:
GAGCAGCAACGAGAAATAAATTGGT;
SEQ. ID. NO. 2:
GCAGACCTGTGTAATGCACATG;
SEQ. ID. NO. 3:
TGTAAGAGTGACCTAAAAACTATACTTATTCTGTTAGA;
SEQ. ID. NO. 4:
CCACTGTCCTTTCTCCTAACAAACT;
SEQ. ID. NO. 5:
CATCATTATCAGGTAGAGGTGACAAGT;
SEQ. ID. NO. 6:
CTCATTGTGTGTTTGTTTTGTCTTCCT;

sequences complementary thereto, sequences which can hybridize under stringent hybridization conditions thereto, and functional discriminatory truncations thereof.

In certain embodiments, the invention extends to a primer or oligonucleotide sets for use in detecting or diagnosing a predisposition to, or increased risk for, developing tendon, ligament, or other soft tissue pathologies or injuries in a subject, the primer or oligonucleotide sets comprising isolated nucleic acid sequences selected from the group consisting of:

Set 1:
SEQ. ID. NO. 1:
GAGCAGCAACGAGAAATAAATTGGT;
SEQ. ID. NO. 2:
GCAGACCTGTGTAATGCACATG;
Set 2
SEQ. ID. NO. 3:
TGTAAGAGTGACCTAAAAACTATACTTATTCTGTTAGA;
SEQ. ID. NO. 4:
CCACTGTCCTTTCTCCTAACAAACT;
Set 3
SEQ. ID. NO. 5:
CATCATTATCAGGTAGAGGTGACAAGT;
SEQ. ID. NO. 6:
CTCATTGTGTGTTTGTTTTGTCTTCCT;
and

sequences complementary thereto, sequences which can hybridize under stringent hybridization conditions thereto, and functional discriminatory truncations thereof.

In addition, the molecular marker comprises, in certain embodiments, any one or more isolated nucleic acid sequences selected from the group consisting of:

SEQ. ID. NO. 7:
CTATTGTTCTCGATTTCT;
SEQ. ID. NO. 8:
ATTGTTCTCAATTTCT;
SEQ. ID. NO. 9:
AACTACTACGACCTCAAAAA;
SEQ. ID. NO. 10:
AACTACTACGACCTCGAAAA;
SEQ. ID. NO. 11:
CAAGGGCTACTTCTAAC;
SEQ. ID. NO. 12:
AAGGGCTACCTCTAAC;
and

fragments thereof, sequences complementary thereto, sequences which can hybridize under stringent hybridization conditions thereto, and functional discriminatory truncations thereof.

Certain embodiments provide an isolated nucleic acid molecule for detecting at least one SNP provided hereinbefore, wherein the nucleic acid molecule comprises less than 40, less than 30, less than 20, or even preferably less than 10 contiguous nucleotides selected from the group consisting of SEQ ID NOS 1 to 12 and fragments, complementary sequences, sequences which can hybridize under stringent hybridization conditions thereto, and functional discriminatory truncations thereof.

### REFERENCES

The following references are incorporated herein by reference only:
1. Puddu G, Ippolito E, Postacchini F. A classification of Achilles tendon disease. Am J Sports Med 1976; 4:145-50.
2. Kader D, Saxena A, Movin T, et al Achilles tendinopathy: some aspects of basic science and clinical management. Br J Sports Med 2002; 36:239-49.
3. Young J S, Kumta S M, Maffulli N. Achilles tendon rupture and tendinopathy: management of complications. Foot Ankle Clin 2005; 10: 371-382.
5 Jarvinen T A, Kannus P, Maffulli N, et al. Achilles tendon disorders: etiology and epidemiology. Foot Ankle Clin 2005; 10: 255-266.
6. Jarvinen T A, Jozsa L, Kannus P, et al. Mechanical loading regulates the expression of tenascin-C in the myotendinous junction and tendon but does not induce de novo synthesis in the skeletal muscle. J Cell Sci 2003; 116(Pt 5):857-866.
7. Jarvinen T A, Jozsa L, Kannus P, et al. Mechanical loading regulates tenascin-C expression in the osteotendinous junction. J Cell Sci 1999; 112 (Pt 18):3157-3166.
8. Mokone G G, Gajjar M, September A V, et al. The guanine-thymine dinucleotide repeat polymorphism within the tenascin-C gene is associated with achilles tendon injuries. Am J Sports Med 2005; 33:1016-1021.
9. Mokone G G, Schwellnus M P, Noakes T D, et al. The COL5A1 gene and Achilles tendon pathology. Scand J Med Sci Sports 2006; 16:19-26.
10. September A V, Cook J, Handley C J, et al. Variants within the COL5A1 gene are associated with achilles tendinopathy in two populations. Br J Sports Med. 2008.
11. Posthumus M, September A V, Schwellnus M P, et al. Investigation of the Sp1-binding site polymorphism within the COL1A1 gene in participants with Achilles tendon injuries and controls. J Sci Med Sport. 2008.
12. September A V, Posthumus M, van der Merwe L, et al. The COL12A1 and COL14A1 genes and Achilles tendon injuries. Int J Sports Med 2008; 29:257-263.
13. Somerville R P, Oblander S A, Apte S S. Matrix metalloproteinases: old dogs with new tricks. Genome Biol. 2003; 4:216.
14. Visse R, Nagase H. Matrix metalloproteinases and tissue inhibitors of metalloproteinases: structure, function, and biochemistry. Circ Res. 2003; 92:827-839.
15. Ye S, Eriksson P, Hamsten A, et al. Progression of coronary atherosclerosis is associated with a common genetic variant of the human stromelysin-1 promoter which results in reduced gene expression. J Biol Chem. 1996; 271:13055-13060.
16. Beyzade S, Zhang S, Wong Y K, et al. Influences of matrix metalloproteinase-3 gene variation on extent of coronary atherosclerosis and risk of myocardial infarction. J Am Coll Cardiol. 2003; 41:2130-2137.
17. Ye S, Patodi N, Walker-Bone K, et al. Variation in the matrix metalloproteinase-3, -7, -12 and -13 genes is associated with functional status in rheumatoid arthritis. Int J. Immunogenet. 2007; 34:81-85.
18. Alfredson H, Lorentzon M, Backman S, et al. cDNA-arrays and real-time quantitative PCR techniques in the investigation of chronic Achilles tendinosis. J Orthop Res. 2003; 21:970-975.
19. Ireland D, Harrall R, Curry V, et al. Multiple changes in gene expression in chronic human Achilles tendinopathy. Matrix Biol. 2001; 20:159-169.
20. Lahiri D K, Nurnberger J I, Jr. A rapid non-enzymatic method for the preparation of HMW DNA from blood for RFLP studies. Nucleic Acids Res. 1991; 19:5444.
22. Barrett J C, Fry B, Mailer J, et al. Haploview: analysis and visualization of LD and haplotype maps. Bioinformatics. 2005; 212:263-265.
23. Chung Y, Lee S Y, Elston R C, Park T. Odds ratio based multifactor-dimensionality reduction method for detecting gene-gene interactions. Bioinformatics. 2007; 23:71-6.
24. September A V, Schwellnus M P, Collins M. Tendon and ligament injuries: the genetic component. Br J Sports Med. 2007; 41:241-246; discussion 246.
25. Magra M, Maffulli N. Genetics: does it play a role in tendinopathy? Clin J Sport Med. 2007; 17:231-233.
26. Jones G C, Corps A N, Pennington C J, et al. Expression profiling of metalloproteinases and tissue inhibitors of metalloproteinases in normal and degenerate human achilles tendon. Arthritis Rheum. 2006; 54:832-842.
27. Riley G P, Curry V, DeGroot J, et al. Matrix metalloproteinase activities and their relationship with collagen remodelling in tendon pathology. Matrix Biol. 2002; 21:185-195.
28. Chard M D, Cawston T E, Riley G P, et al. Rotator cuff degeneration and lateral epicondylitis: a comparative histological study. Ann Rheum Dis. 1994; 53:30-34.
29. Pierfitte C, Royer R J. Tendon disorders with fluoroquinolones. Therapie. 1996; 51:419-420.
30. Corps A N, Harrall R L, Curry V A, et al. Ciprofloxacin enhances the stimulation of matrix metalloproteinase 3 expression by interleukin-1 beta in human tendon-derived cells. A potential mechanism of fluoroquinolone-induced tendinopathy. Arthritis Rheum. 2002; 46:3034-3040.
31 Voet D, Voet J G, Pratt C W. Fundamentals of Biochemistry (Upgraded Edition). Hoboken, N.J.: Wiley 2002:77-81.
32. Riva A, Kohane I S. A SNP-centric database for the investigation of the human genome. BMC Bioinformatics. 2004; 5:33.
33. Becker J W, Marcy A I, Rokosz L L, et al. Stromelysin-1: three-dimensional structure of the inhibited catalytic domain and of the C-truncated proenzyme. Protein Sci. 1995; 4:1966-1976.
34. Nagase H. Activation mechanisms of matrix metalloproteinases. Biol Chem. 1997; 378:151-160.
35. September A V, Schwellnus M P, Collins M. Tendon and ligament injuries: the genetic component. Br J Sports Med. 2007; 41:241-246
36. Birkedal-Hansen H, Moore W G, Bodden M K, et al. Matrix metalloproteinases: a review. Crit Rev Oral Biol Med. 1993; 4:197-250.

TABLE 1
PCR oligonucleotides and probes used for geno-
typing. Primer and probe sets were incorporated
into a PCR mastermix and used as described in
materials and methods.

| | Oligonucleotides | |
|---|---|---|
| SNP | (forward/reverse) | Probes |
| rs591058 | GAGCAGCAACGAGAAATAAA | VIC-CTATTGTTCTCGAT |
| | TTGGT | TTCT |
| | GCAGACCTGTGTAATGCACA | FAM-ATTGTTCTCAATTT |
| | TG | CT |
| rs679620 | TGTAAGAGTGACCTAAAAAC | VIC-AACTACTACGACCT |
| | TATACTTATTCTGTTAGA | CAAAAA |
| | CCACTGTCCTTTCTCCTAAC | FAM-AACTACTACGACCT |
| | AAACT | CGAAAA |
| rs650108 | CATCATTATCAGGTAGAGGT | VIC-CAAGGGCTACTTCT |
| | GACAAGT | AAC |
| | CTCATTGTGTGTTTGTTTTG | FAM-AAGGGCTACCTCTA |
| | TCTTCCT | AC |

TABLE 2
Characteristics of the control (CON), Achilles tendinopathy (TEN) and Achilles
rupture (RUP) subjects.

| | CON | TEN | RUP | P value^{b} |
|---|---|---|---|---|
| Age (years)^{a} | 36.8 ± 9.9 (91) | 40.5 ± 13.7 (70) | 40.7 ± 11.5 (37) | 0.078 |
| Height (cm) | 176 ± 10 (95) | 177 ± 9 (66) | 175 ± 8 (37) | 0.623 |
| Weight (kg) | 72.0 ± 12.1 (97) | 78.4 ± 14.1 (69) | 85.2 ± 15.4 (37) | <0.001^{c} |
| BMI (kg/cm²) | 23.3 ± 2.8 (95) | 24.9 ± 3.4 (66) | 27.8 ± 3.7 (37) | <0.001^{c} |
| Gender (% males) | 67.0 (97) | 73.0 (74) | 73.0 (37) | 0.644 |
| Country of birth (% | 74.2 (97) | 73.2 (71) | 78.4 (37) | 0.837 |
| South Africa) | | | | |

Gender and country of birth are summarised as a percentage (%), while the remaining variables are expressed as mean ± standard deviation. The number of subjects (N) is in parenthesis.
BMI—body mass index.
^{a}The age of the TEN and RUP groups are the age of the onset of the initial symptoms of Achilles tendon injury, which were on average 7.8 ± 8.0 and 7.5 ± 8.9 years prior to recruitment in this study respectively.
^{b}P value = Global, so CON vs TEN vs RUP.
^{c}Except for the weight difference between the TEN and RUP groups (P = 0.033), all other pairwise differences in weight and BMI were highly significant (P < 0.01).

TABLE 3
The MMP3 rs679620 G/A and COL5A1 rs12722 C/T genotype
pairs, together with their frequencies within the chronic Achilles
tendinopathy (TEN) and control (CON) groups, as well as their
estimated risk (OR) and the risk order.

| | | CON | TEN | | Risk |
|---|---|---|---|---|---|
| MMP3 | COL5A1¹ | (N = 98) | (N = 74) | OR | Order |
| GG | TT | 3.1 (3) | 10.8 (8) | 3.48 | 8 |
| GG | TC | 12.2 (12) | 20.3 (15) | 1.66 | 6 |
| GG | CC | 4.1 (4) | 6.8 (5) | 1.66 | 7 |
| AG | TT | 21.4 (21) | 23.0 (17) | 1.07 | 5 |
| AG | TC | 16.3 (16) | 16.2 (12) | 0.99 | 4 |
| AG | CC | 18.4 (18) | 4.1 (3) | 0.22 | 1 |
| AA | TT | 1.0 (1) | 5.4 (4) | 5.40 | 9 |
| AA | TC | 15.3 (15) | 9.5 (7) | 0.62 | 3 |
| AA | CC | 8.2 (8) | 4.1 (3) | 0.50 | 2 |

The CON and TEN values are represented as a frequency (%) with the number of subjects (N) in parenthesis.
¹The CC genotype is under-represented in TEN subjects [10].

1. A method of determining in a subject a predisposition to, or increased risk for, developing a tendon, ligament, or other soft tissue injury or pathology, the method comprising the step of screening the subject for the presence of at least one polymorphism n at least one member of the matrix metallo-protease (MMP) gene family, which polymorphism is a polymorphism that results in a modified, augmented, or mitigated interaction of the gene and/or gene product with the genes and/or gene products of other members of the matrix metallo-protease, collagen or extracellular matrix glycoprotein gene families, when compared to a wild-type interaction.

2. The method of claim 1, which includes the step of screening the subject for the presence of at least one polymorphism in at least one member of at least one gene family selected from the group consisting of any one or more of
the collagen gene family, including the COL5A1 and COL12A1 genes;
the extracellular matrix glycoprotein gene family, including the TNC and COMP genes; and derivatives thereof.

3. The method of claim 1, which includes the step of detecting or screening for the presence of a polymorphism in the matrix metallo-protease 3 (MMP3) gene which polymorphism results in a modified, augmented, or mitigated interaction with a COL5A1 polymorphism gene and/or gene product, when compared to a wild-type interaction.

4. The method of claim 3, wherein the MMP3 polymorphism is a polymorphism which results in a modified, augmented, or mitigated interaction with the rs12733 COL5A1 polymorphism, and/or any other genetically linked polymorphism, and/or the product encoded thereby.

5. The method of claim 1, which includes the additional steps of:
providing a tissue sample from a subject;
extracting nucleic acid from the sample;
amplifying selected regions of the nucleic acid using any one or more of the primers selected from the group consisting of: SEQ. ID. NOs 1 to 2, thereby to obtain amplified nucleic acid fragments; and
screening the amplified nucleic acid fragments for the presence of the polymorphisms of claim 1.

6. A molecular probe or primer for use in diagnosing a predisposition to, or increased risk for, developing tendon, ligament, or other soft tissue pathology or injury in a subject, the molecular probe or primer comprising at least one isolated nucleic acid fragment of between 10 and 40 nucleotides derived from an MMP3 gene, flanking sequences thereof, cis-regions associated therewith, 5′UTR regions thereof, and 3′UTR regions thereof, sequences complementary thereto, sequences which can hybridize under strict hybridization conditions thereto, and functional discriminatory truncations thereof, which molecular probe or primer hybridizes under stringent hybridization conditions to at least a portion of the MMP3 gene, sequences complementary to the probe, and sequences having substitutions, deletions or insertions, sequences which can hybridize under strict hybridization conditions thereto, and functional discriminatory truncations thereof.

7. The molecular probe or primer of claim 6, which is between 15 and 35 nucleotides in length.

8. The molecular probe or primer of claim 7, which is between 20 and 30 nucleotides in length.

9. The molecular probe or primer of claim 6, which incorporates a single nucleotide polymorphism (SNP) selected from the group consisting of any one or more of rs591058, rs679620, rs650108, and other SNPs in high linkage disequilibrium with rs591058, rs679620, or rs650108.

10. The molecular probe or primer of claim 9, wherein the incorporated SNP is selected from the group consisting of:
(i) rs679620, an A/G transition at nucleotide position 28 within exon 2, E45K;
(ii) rs591058, a T/C transition at nucleotide position 1547 within intron 4; and
(iii) rs650108, a G/A transition at position 495 within intron 8.

11. The molecular probe or primer of claim 6, which comprises, or is detectable using, any one or more oligonucleotides selected from the group comprising SEQ. ID. NOs 1 to 6, sequences complementary thereto, sequences which can hybridize under stringent hybridization conditions thereto, and functional discriminatory truncations thereof.

12. The molecular probe or primer of claim 6, which comprises any one or more sequences selected from the group consisting of: SEQ. ID. NOs 7 to 12, fragments thereof, sequences complementary thereto, sequences which can hybridize under stringent hybridization conditions thereto, and functional discriminatory truncations thereof.

13. A primer or oligonucleotide set for use in detecting or diagnosing a predisposition to, or increased risk for, developing tendon, ligament, or other soft tissue pathologies or injuries in a subject, the primer or oligonucleotide set being any one or more sets of isolated oligonucleotide sequences selected from the group consisting of:
Set 1: SEQ. ID. NO. 1 and 2;
Set 2: SEQ. ID. NO. 3 and 4;
Set 3: SEQ. ID. NO. 5 and 6; and
sequences complementary thereto, sequences which can hybridize under stringent hybridization conditions thereto, and functional discriminatory truncations thereof.

14. An isolated nucleic acid molecule for detecting at least one SNP as claimed in claim 9, wherein the nucleic acid molecule comprises less than 30 contiguous nucleotides selected from the group consisting of SEQ. ID. NOs. 1 to 12, fragments, complementary sequences, sequences which can hybridize under stringent hybridization conditions thereto, and functional discriminatory truncations thereof.

15. The isolated nucleic acid molecule of claim 14, which comprises less than 20 contiguous nucleotides selected from the group consisting of SEQ. ID. NOs. 1 to 12, fragments, complementary sequences, sequences which can hybridize under stringent hybridization conditions thereto, and functional discriminatory truncations thereof.

16. The isolated nucleic acid molecule of claim 14, which comprises less than 10 contiguous nucleotides selected from the group consisting of SEQ. ID. NOs. 1 to 12, fragments, complementary sequences, sequences which can hybridize under stringent hybridization conditions thereto, and functional discriminatory truncations thereof.

17. A diagnostic assay comprising any one or more of the molecular probes or primers claimed in claim 6, fragments thereof, sequences complementary thereto, sequences which can hybridize under stringent hybridization conditions thereto, and functional discriminatory truncations thereof.

18. A method for diagnosing a predisposition to a soft tissue pathology in a subject using the molecular probe or primer as claimed in claim 6, the method comprising;
providing the molecular probe or primer, which comprises at least one isolated nucleic acid fragment of between 10 and 40 nucleotides derived from an MMP3 gene, flanking sequences thereof, cis-regions associated therewith, 5′UTR regions thereof, and 3′UTR regions thereof, sequences complementary thereto, sequences which can hybridize under strict hybridization conditions thereto, and functional discriminatory truncations thereof; and
hybridizing the molecular probe or primer under stringent hybridization conditions to at least a portion of the MMP3 gene, sequences complementary to the probe, and sequences having substitutions, deletions or insertions, sequences which can hybridize under strict hybridization conditions thereto, and functional discriminatory truncations thereof.

19. A kit for use in diagnosing a predisposition to a soft tissue pathology in a subject, the kit comprising:
any two or more of the molecular probes or primers selected from the group consisting of:
SEQ. ID. Nos 1 to 12; and
suitable reaction media.

20. The kit of claim 19, which further includes any one or more of reagents, buffers, DNases, RNAses, polymerases, and instructions.

21. The method of claim 1, wherein the soft tissue is a connective tissue injury, and includes any one of tendon and ligament injuries.

22-27. (canceled)
